(19) United States
(12) Patent Application Publication
(10) Pub. No.: US 2022/0201830 A1
(43) Pub. Date: Jun. 23, 2022

(54) X-RAY SOURCE ASSEMBLY WITH ENHANCED TEMPERATURE CONTROL FOR OUTPUT STABILITY

(71) Applicant: X-RAY OPTICAL SYSTEMS, INC., East Greenbush, NY (US)

(72) Inventors: Ernest COOLEY, Henrietta, NY (US); Xin ZHANG, Latham, NY (US); Fuzhong WEI, Altamont, NY (US)

(73) Assignee: X-RAY OPTICAL SYSTEMS, INC., East Greenbush, NY (US)

(21) Appl. No.: 17/132,094

(22) Filed: Dec. 23, 2020

(51) Int. Cl. H05G 1/36; H05G 1/32

(52) U.S. Cl. CPC H05G 1/36; A61B 6/4488; H05G 1/32

(57) ABSTRACT

An x-ray source assembly includes an anode stack including a source spot upon which electrons impinge with power being supplied to the assembly, and a control system to facilitate maintaining intensity of output x-rays from the x-ray source assembly during operation. The control system is configured to actively control temperature of the anode stack relative to a setpoint or defined setpoint range. The control system heats the anode stack in a heating mode, when an anode stack temperature is below the setpoint or defined setpoint range, and switches to a cooling mode to cool the anode stack when the anode stack temperature rises above the setpoint or defined setpoint range.

### BACKGROUND

Small, compact x-ray tubes have experienced widespread adoption in instruments for x-ray fluorescence (XRF) spectroscopy and x-ray diffraction (XRD) for a wide range of industrial, medical and dental applications. X-ray tubes conventionally emit radiation in a divergent manner. Obtaining an illumination spot size of sufficient intensity typically necessitated expensive, high-powered sources. The recent ability to focus x-ray radiation has enabled reductions in the size and cost of x-ray sources, and hence x-ray systems have been adopted in a variety of applications. X-ray beam production and transmission is exemplified by the polycapillary focusing and collimating optics and the in optic/source combinations such as those disclosed in commonly assigned, X-Ray Optical Systems, Inc. U.S. Pat. Nos. 5,192,869; 5,175,755; 5,497,008; 5,745,547; 5,570,408; 5,604,353; 6,781,060; 7,110,506; and 7,257,193, all of which are hereby incorporated by reference herein in their entirety.

### SUMMARY

The present invention relates generally to x-ray sources, and more particularly, to x-ray source assemblies having a focused or collimated or redirected x-ray beam output with enhanced stability. While progress in x-ray focusing has recently been achieved, further enhancements to x-ray source assemblies are still desired. For example, improving the output stability of an x-ray beam under changing operating conditions and changing operating modes is desired.

The shortcomings of the prior art are overcome and additional advantages are provided through the provision, in one or more aspects, of an x-ray source assembly which includes an anode stack, and a control system. The anode stack has a source spot upon which electrons impinge based on power supplied to the assembly, and the control system facilitates maintaining intensity of output x-rays during operation of the x-ray source assembly. The control system is configured to actively control temperature of the anode stack relative to a setpoint or defined setpoint range. The control system heats the anode stack in a heating mode, when an anode stack temperature is below the setpoint or defined setpoint range, and switches to a cooling mode to cool the anode stack when the anode stack temperature rises above the setpoint or defined setpoint range.

Further, additional features and advantages are realized through the techniques of the present invention. Other embodiments and aspects of the invention are described in detail herein and are considered a part of the claimed invention.

### BRIEF DESCRIPTION

The subject matter regarded as the invention is particularly pointed out and distinctly claimed in the claims at the conclusion of the specification. The foregoing and other objects, features, and advantages of the invention are apparent from the following detailed description taken in conjunction with the accompanying drawings in which:

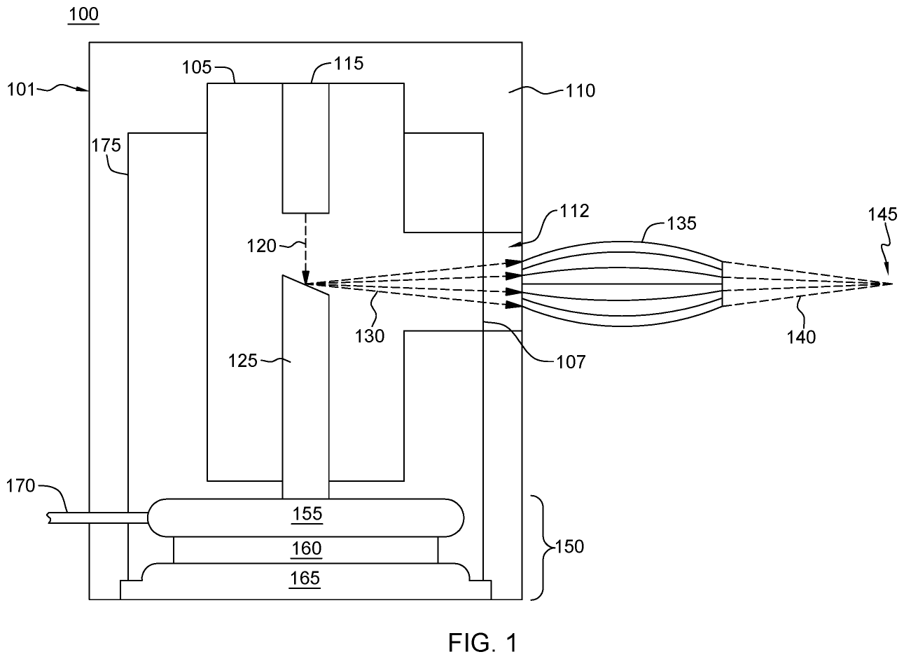

FIG. 1 depicts a cross-sectional view of one embodiment of an x-ray source assembly, in accordance with one or more aspects of the present invention;

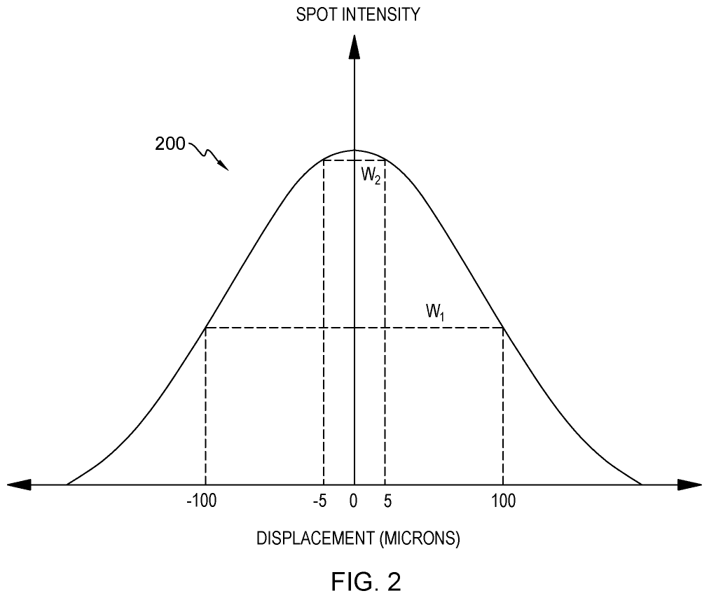

FIG. 2 depicts one example of a source scan curve for an x-ray source such as shown in FIG. 1 plotting output intensity versus displacement, in accordance with an aspect of the present invention;

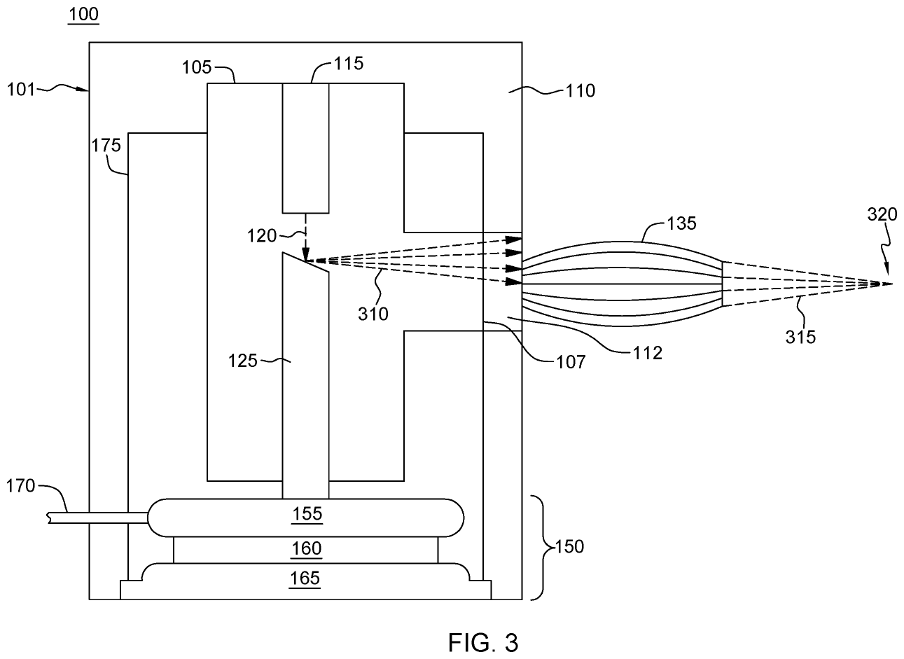

FIG. 3 depicts a cross-sectional view of the x-ray source assembly of FIG. 1 showing a source spot to optic misalignment, which is addressed in accordance with one or more aspects of the present invention;

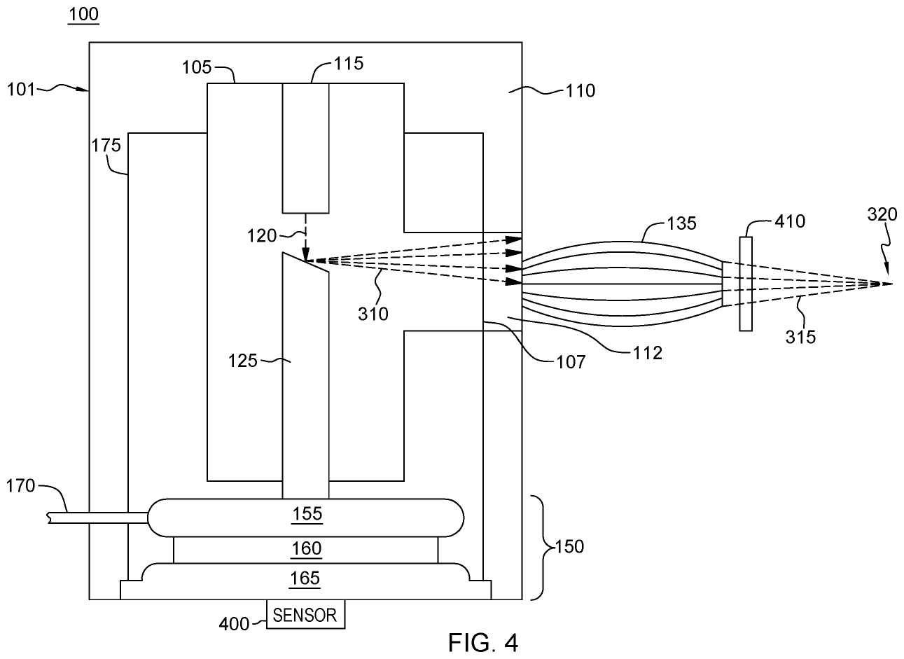

FIG. 4 depicts a cross-sectional view of the x-ray source assembly of FIG. 3 showing different sensor placements for monitoring source spot to optic displacement, in accordance with an aspect of the present invention;

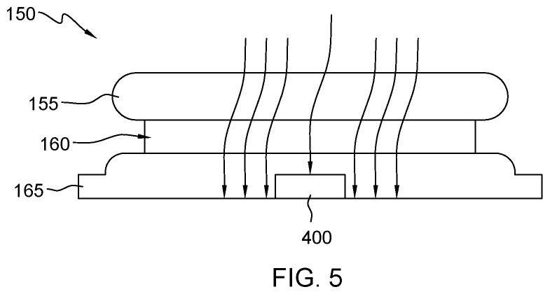

FIG. 5. is a cross-sectional view of one embodiment of the anode base assembly of the anode stack depicted in FIGS. 1, 3 & 4, in accordance with an aspect of the present invention;

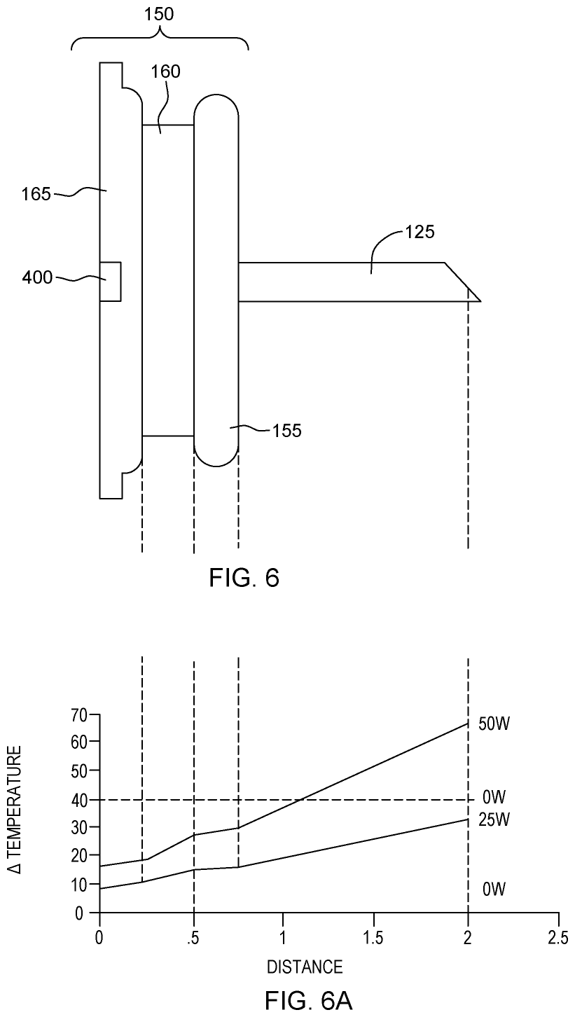

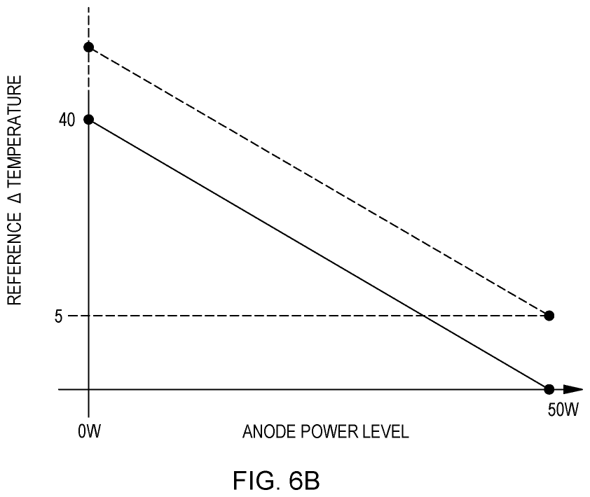

FIG. 6 is a cross-sectional view of the anode stack embodiment of FIGS. 1, 3 & 4, in accordance with an aspect of the present invention;

FIG. 6A is a graphical representation of change in temperature across the elements of the anode stack for different anode power levels, in accordance with an aspect of the present invention;

FIG. 6B is a graph of change in reference temperature as a function of anode power level, in accordance with an aspect of the present invention;

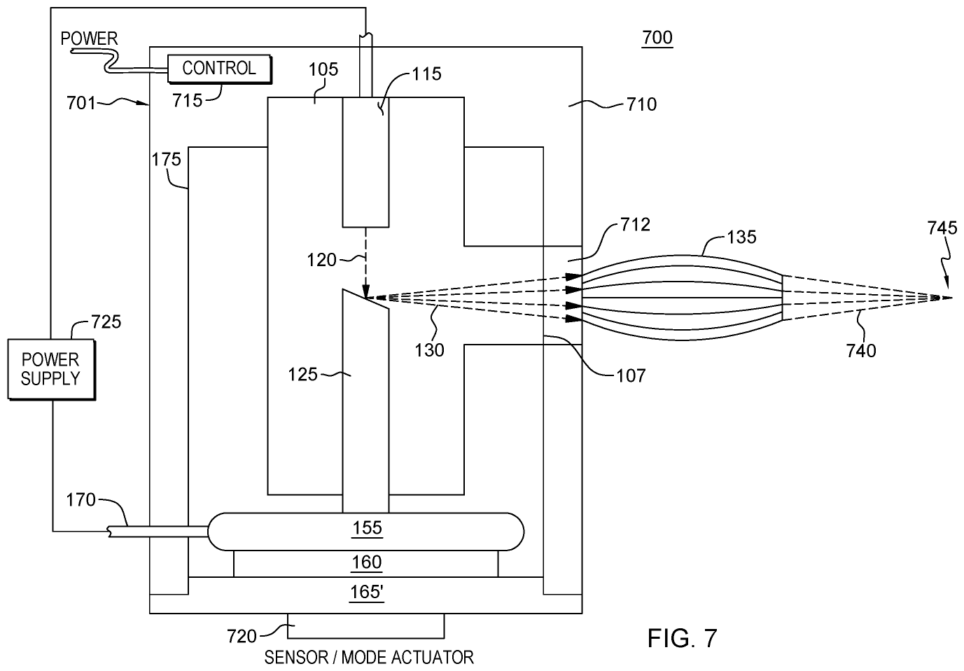

FIG. 7 depicts a cross-sectional view of one embodiment of an enhanced x-ray source assembly, in accordance with one or more aspects of the present invention;

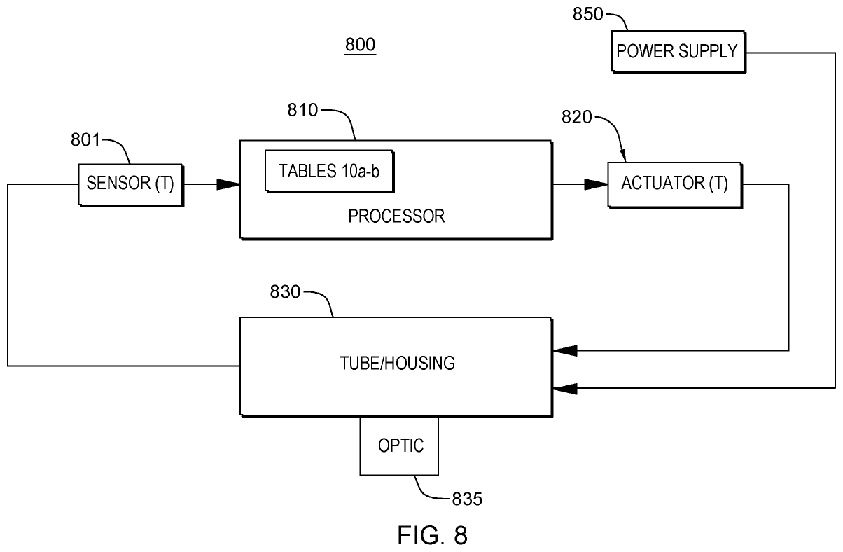

FIG. 8 depicts a block diagram of one embodiment of a control system for an x-ray source assembly, in accordance with an aspect of the present invention;

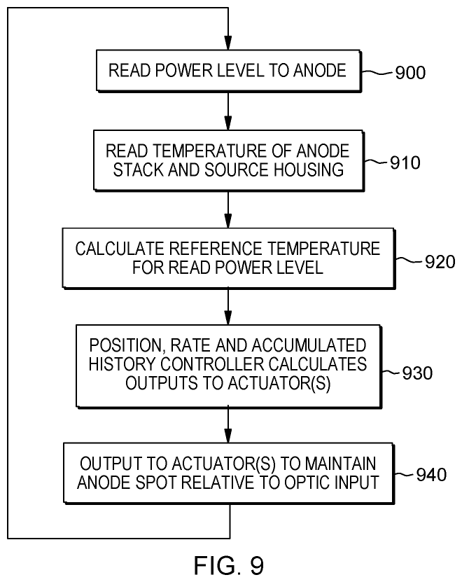

FIG. 9 is a flowchart of one embodiment of control processing for an x-ray source assembly, in accordance with aspects of the present invention;

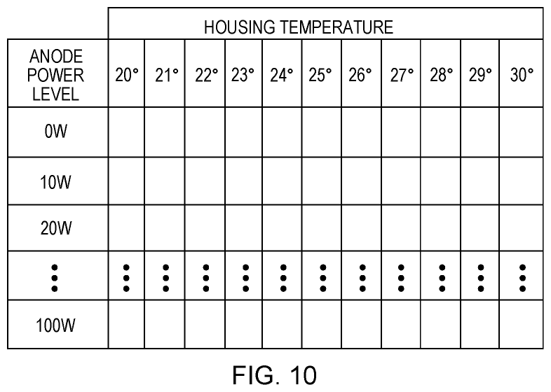

FIG. 10 is an exemplary reference temperature table which can be employed by one embodiment of the control system processing of FIG. 9, in accordance with aspects of the present invention;

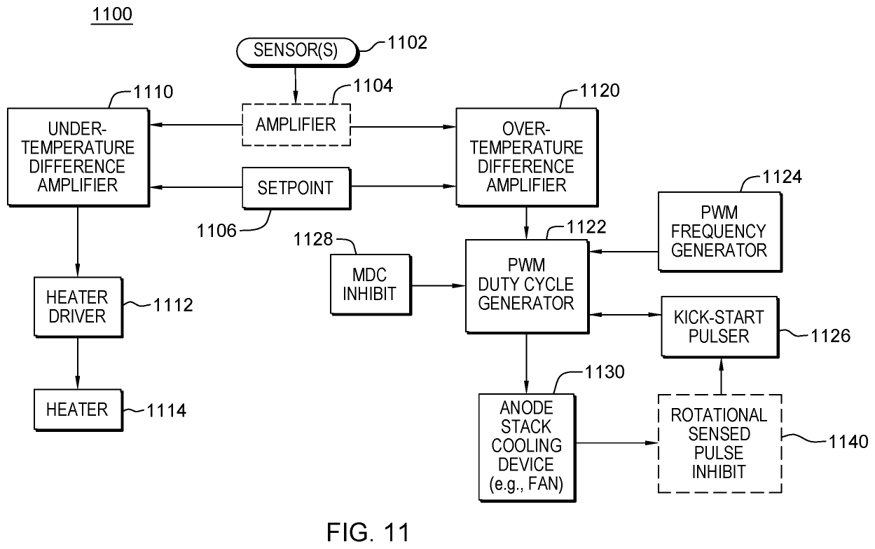

FIG. 11 is a block diagram of another embodiment of an x-ray source assembly control system, in accordance with one or more aspects of the present invention;

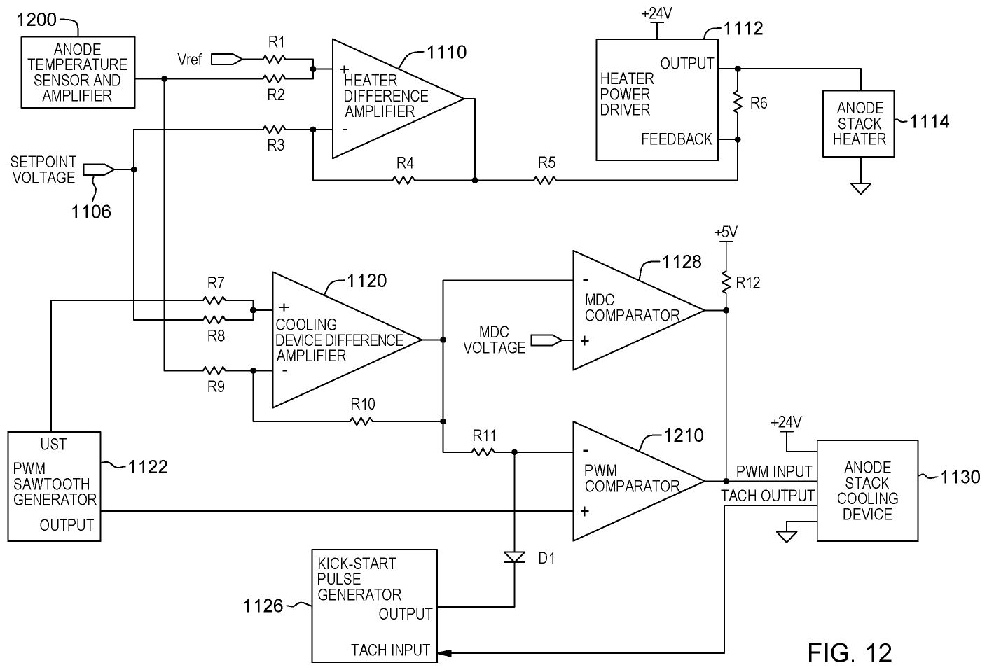

FIG. 12 is a schematic of a more detailed embodiment of the x-ray source assembly control system of FIG. 11, in accordance with one or more aspects of the present invention; and

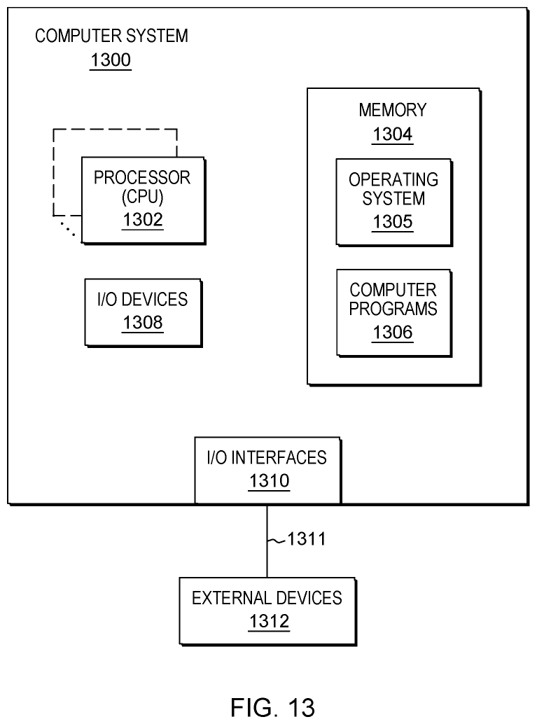

FIG. 13 depicts one embodiment of a computer system and associated devices to incorporate and/or use one or more control aspects of an x-ray source assembly control system embodiment, in accordance with one or more aspects of the present invention.

### DETAILED DESCRIPTION

As generally discussed above, the present invention provides in one aspect an x-ray source assembly providing, for example, a focused x-ray beam, a collimated x-ray beam, or a redirected x-ray beam, and having a stable output over a range of operating conditions. This stable output is obtained via a control system which controls, in one aspect, anode stack temperature, notwithstanding a change in one or more of the operating conditions, or a change in operating mode.

The control system includes one or more actuators which can effect the necessary changes. For example, one actuator is a temperature actuator which provides heating (in a heating mode) or cooling (in a cooling mode) of the anode stack to effect control of the anode source spot location relative to the output structure. One or more sensors can be employed by the control system, including one or more temperature sensors, such as a sensor to directly or indirectly measure the anode stack temperature, as well as a housing temperature sensor and an ambient temperature sensor, if desired.

As used herein, the phrase “output structure” refers to a structure including part of the x-ray source assembly or associated with the x-ray source assembly. By way of example, the structure could include an x-ray transmission window or an optic, such as a focusing or collimating optic, which may or may not be secured to a housing surrounding the x-ray tube within the assembly.

FIG. 1 illustrates in cross-section an elevational view of an x-ray source assembly 100 in accordance with an aspect of the present invention. X-ray source assembly 100 includes an x-ray source 101 including a vacuum tight x-ray tube 105 (typically formed of glass or ceramic) having a transmission window 107. X-ray tube 105 houses an electron gun 115 arranged opposite a high-voltage (HV) anode 125. When voltage is applied, electron gun 115 emits electrons in the form of an electron stream, i.e., an electron beam (e-beam) 120, as is well known in the art. HV anode 125 acts as a target with a source spot upon which the electron stream impinges for producing x-ray radiation, i.e., x-rays 130.

By way of example, electron gun 115 can be held at ground potential (zero volts), while HV anode 125 is held at a high voltage potential, typically around 50kv. As a result, e-beam 120 emitted from electron gun 115 at ground potential is electrically attracted to the surface of HV anode 125, thereby producing x-rays 130 from a source spot on the anode where e-beam 120 strikes the anode. X-rays 130 are subsequently directed through transmission window 107 of vacuum tight x-ray tube 105. Transmission window 107 is typically formed of a material such as beryllium (Be) which permits substantially unimpeded transmission of x-rays while still maintaining the vacuum within x-ray tube 105.

A housing 110 at least partially encloses x-ray tube 105. Housing 110 can include an aperture 112 aligned with transmission window 107 of x-ray tube 105. By way of example, aperture 112 can be an open aperture in housing 110 or an enclosed aperture defining an air space. Upon transmission through transmission window 107 and aperture 112, x-rays 130 are collected by an optic 135. Optic 135 is shown in this example centered about aperture 112 in housing 110. Optic 135 can be affixed to an exterior surface of housing 110, or be partially disposed within housing 110 to reside within aperture 112 (e.g., to reside against transmission window 107), or can be separately supported from housing 110 but aligned to aperture 112 in housing 110.

As noted, optic 135 can include a focusing optic, a collimating optic, or a redirecting optic, by way of example. In FIG. 1, optic 135 is shown to be a focusing element, which is useful when x-ray source 100 is utilized for applications requiring a high intensity, low diameter spot 145. Focusing optic 135 collects x-ray radiation 130 and focuses the radiation into converging x-rays 140. A focusing optic is beneficial when x-ray source 100 is to be employed in connection with an x-ray fluorescence system which requires a low power source. As an alternative, optic 135 can include a collimating optical element for use in applications which require a parallel beam of x-ray radiation output from the optic (not shown). In the case of a collimating optical element, x-rays 140 are parallel rather than converging to spot 145 as shown in FIG. 1.

Optic 135 can include any optical element capable of collecting or manipulating x-rays, for example, for focusing or collimating. By way of example, optic 135 can be a polycapillary bundle (such as available from X-ray Optical Systems, Inc. of Albany, New York), a doubly curved optic or other optical element form, such as a filter, a pinhole or a slit. (A polycapillary optic is a bundle of thin, hollow tubes that transmit photons via total reflection. Such an optic is described, for example, in U.S. Pat. Nos. 5,175,755, 5,192,869, and 5,497,008. Doubly curved optics are described, for example, in U.S. Pat. Nos. 6,285,506 and 6,317,483. All of these patents are incorporated by reference herein in their entirety.) Upon calibration of x-ray source assembly 100, optic 135 remains stationary (in one embodiment) relative to x-ray source 101 until further calibration of x-ray source assembly 100 is performed.

The end of HV anode 125 opposite the impingement surface protrudes through the body of x-ray tube 105 and is mechanically and electrically connected to a base assembly 150. Base assembly 150 includes a first conductor disc 155 that is electrically isolated from a base plate 165 via a dielectric disc 160. One embodiment of the resulting anode 125 and base assembly 150 structure, referred to herein as the anode stack, is described in detail in the above-incorporated U.S. Pat. No. 7,110,506 B2, entitled “Method and Device For Cooling and Electrically Insulating A High-Voltage, Heat Generating Component Such as an X-Ray Tube for Analyzing Fluid Streams”.

In one or more embodiments, conductor disc 155 and base plate 165 are, for example, several inches in diameter, disc-shaped plates formed of a highly electrically conductive and highly thermally conductive material, such as copper. By way of example, conductor disc 155 and base plate 165 can have a thickness in the range of 0.1 to 0.5 inches, with 0.25 inches being one specific example. Base plate 165 can further include constructional detail to accommodate the overall structure of x-ray source 101.

Dielectric disc 160 is, for example, a 1.5-inch diameter, disc-shaped plate formed of a material that provides high dielectric strength at high voltages, such as ceramic. In addition, while not as thermally conductive as conductor disc 155 or base plate 165, these materials do exhibit relatively good thermal conductivity.

Conductor disc 155 is mechanically and electrically connected to a high voltage source (not shown) via an appropriate high voltage lead 170. As a result, the high voltage potential is supplied to conductor disc 155 and subsequently to HV anode 125. Conversely, base plate 165 is held at ground potential. Dielectric disc 160 provides electrical isolation between high-voltage conductor disc 155 and the grounded base plate 165. One example of an assembly for connecting high voltage lead 170 to conductor disc 155 is described in the above-incorporated U.S. Pat. No. 6,781,060 B2, entitled “An Electrical Connector, A Cable Sleeve, and A Method For Fabricating A High Voltage Electrical Connection For A High Voltage Device”.

The x-ray tube 105, base assembly 150, and HV lead 170, can be encased in an encapsulant 175. Encapsulant 175 can be, or include, a rigid or semi-rigid material with a sufficiently high dielectric strength to avoid voltage breakdown, such as silicone. Furthermore, encapsulant 175 need not be a good thermal conductor since the preferred thermal path is through base assembly 150.

FIG. 2 graphically illustrates a source scan curve 200 in which a representation of output intensity, e.g., spot 145 (FIG. 1) intensity, is plotted with respect to displacement or misalignment between the anode source spot and the output optic. The spot intensity results from scanning x-rays (130) across the focal point of optic (135). It is shown that a Gaussian plot results, in which a maximum intensity is achieved with proper alignment of x-rays 130 (and thus the anode source spot) at the focal point of the optic.

As shown, the full width W1 at half maximum (FWHM) is equal to approximately 200 microns. A FWHM of 200 microns indicates that the x-ray intensity at spot 145 drops 50% as a result of displacement of x-rays 130 (and thus the anode source spot) a distance of 100 microns from the focal point of optic 135. When properly calibrated, x-ray source assembly 100 functions for a given power near the top of the source scan curve of FIG. 2, where the slope is approximately equal to zero, such that minor perturbations in the displacement of x-rays 130 (e.g., 5 micrometers or less) with respect to optic 135 result in a negligible intensity drop. By way of example, the range of allowable perturbations in the displacement of x-rays 130 with respect to optic 135 is represented by W2, indicating that a displacement less than five microns between x-rays 130 and the focal point of optics 135 is acceptable.

FIG. 3 depicts x-ray source 100 as described above in connection with FIG. 1. In this example, however, heat generated by e-beam 120 impinging on HV anode 125 has caused HV anode 125, conductor disc 155, base plate 165, and to a lesser extent, dielectric disc 160, to expand. As a result of this expansion, a divergent beam of x-rays 310 is generated that is displaced vertically with respect to x-rays 130 illustrated in FIG. 1. For example, if the x-ray tube or target of electron gun 115 are operated at a power of 50 W, then the focal point of x-rays 310 may be displaced by as much as 50 microns from its position at 0 W. X-rays 310 are misaligned with optic 135 and, as a result, the convergent beam of x-rays 315 produces a spot 320 of markedly reduced intensity.

Other environmental conditions can cause this displacement. As discussed below, the present invention is related to compensating for this displacement by dynamically controlling temperature of the anode stack.

Due to the physical nature of collimating optics and focusing optics, such as doubly curved crystals and polycapillary bundles, precise positioning of optic 135 relative to the anode source spot is desirable for optimum collimation or focusing of x-rays 315. As a result, a displacement of x-rays 310 with respect to optic 135 such as can result from thermal expansion of HV anode 125 and base assembly 150 can result in a spot 320 having significantly reduced intensity, as illustrated graphically in FIG. 2.

The anode source spot to an output structure offset can be controlled using various approaches. For example, as depicted in FIG. 4, a temperature sensor 400 can be employed at the base of the anode stack to measure used to control the anode stack temperature relative to a setpoint temperature. FIG. 5 shows an alternative temperature sensor implementation.

As shown in FIG. 5, base assembly 150, again including conductor disc 155, dielectric disc 160 and base plate 165, is modified to include a temperature sensor 400 recessed within and in good thermal contact with base plate 165. For illustrative purposes, FIG. 5 depicts waves which represent heat transfer from the anode, to and through the base assembly. These waves represent heat which is generated by the impingement of e-beam 120 upon HV anode 125 as shown in FIG. 4.

Also depicted in FIG. 4 is an x-ray intensity measurement device 410. In addition to sensing temperature to determine offset, x-ray output intensity of either x-ray source 101 or optic 135 could be measured. By way of example, in a diffraction application, an ion chamber or a proportional counter could be used as an intensity measurement device 410 in order to provide the needed feedback for a temperature control system such as described herein. In a diffraction application, the energy of interest is typically only at one wavelength and thus a proportional counter disposed within the x-ray path only absorbs a small amount of the x-rays of interest. Those skilled in the art will recognize that other intensity measurement approaches can be employed to directly or indirectly determine the intensity of x-rays output from the x-ray source assembly 100. The goal of temperature sensing, x-ray intensity sensing, etc., is to provide supplemental feedback information on the alignment between the anode source spot and the output structure. A control system and a control process are described further below with reference to FIGS. 7-13.

The correlation between anode stack temperature and anode source spot to output structure alignment can be better understood with reference to FIGS. 6-6B.

In FIG. 6, one embodiment of an anode stack is shown to include anode 125 and base assembly 150. Assembly 150 includes, in one embodiment, conductor disc 155, dielectric disc 160 and base plate 165, which in this example is shown with temperature sensor 400 embedded therein. The anode stack is positioned horizontally in order to correlate with the distance axis (x-axis) on the graph of FIG. 6A.

As shown in FIG. 6A, the anode stack has different temperature drops across the various components of the stack. Beginning at the right most end of anode 125, for both a 50 W and 25 W example, there is shown a temperature drop which has a slope slightly steeper than the temperature drop across, for example, conductor disc 155. Although both anode 125 and disc 155 are conductive, the larger cross-section for disc 155 means that there is less of a temperature drop from one main surface to the other. Also as shown in FIG. 6A, the change in temperature across the anode stack can relate to the anode power level in operation. The change in temperature (y-axis) refers to a changing temperature offset of the anode stack above room temperature. Thus, at zero applied anode power level the offset is assumed to be zero.

As a further enhancement, an x-ray source assembly in accordance with an aspect of the present invention can be adjusted to accommodate for changes in room or ambient temperature. In order for the total thermal expansion of the elements contributing to the expansion to be the same at 50 W beam current as at 0 W beam current, then the 0 W base temperature of plate 165 (and hence the connected elements) can be raised to, for example, 40 degrees C. This is shown in FIG. 6A by the dotted line.

FIG. 6B depicts an example of reference temperature less ambient temperature of a component of the anode stack for various anode power levels between 0 and 50 Watts. More particularly, FIG. 6B depicts the reference temperature (derived and shown at 0 W in FIG. 6A) for various tube operating powers. Further, by adding an additional temperature offset to this reference temperature, the same system can accommodate changes in ambient temperature. For example, at 50 W and 20 C, a 0 C reference delta temperature is obtained. If this reference delta temperature is raised to 5 C, then additional heating is to be supplied to maintain this delta temperature at 20 C. However at 25 C, no additional heating is required. In this way, an offset in the reference delta temperature is required at, for example, 20 C, which allows for compensation at higher ambient temperatures.

FIG. 7 illustrates in cross-section an elevational view of one embodiment of an x-ray source assembly, generally denoted 700, in accordance with further aspects of the present invention. X-ray source assembly 700 includes an x-ray source 705 and an output optic 135. Optic 135 is aligned to x-ray transmission window 107 of vacuum x-ray tube 105. X-ray tube 105 again houses electron gun 115 arranged opposite to high voltage anode 125. When voltage is applied, electron gun 115 emits electrons in the form of an electron stream (i.e., electron beam 120 as described above). HV anode 125 acts as a target with respect to a source spot upon which the electron stream impinges for producing x-ray radiation 130 for transmission through window 107 and collection by optic 135. Electron gun 115 and anode 125 function as described above in connection with the embodiments of FIGS. 1, 3 & 4.

Anode 125 is physically and electrically connected to a base assembly which includes a conductor plate 155 that is electrically isolated from a base plate 165 via a dielectric disc 160. In one embodiment, the construction and function of the base assembly can be similar to the base assemblies described above in connection with FIGS. 1, 3 & 4. High voltage leads 170 connect to conductive plate 155 to provide the desired power level to anode 125. The electron gun 115, anode 125, base assembly 150 and high voltage lead 170 are encased by encapsulant 175 all of which reside within a housing 710. Housing 710 includes an aperture 712 aligned to x-ray transmission window 107 of x-ray tube 105. In operation, x-ray radiation 130 is collected by optic 135, and in this example, focused 740 to a spot 745. As noted above, optic 135 can be, or include, any one of various types of optical elements, including polycapillary bundles and doubly curved crystals. Also, optic 135 can be, for example, a focusing optic or a collimating optic depending upon the application for the x-ray source assembly.

In accordance with an aspect of the present invention, a control system is implemented for x-ray source assembly 700. This control system includes, for example, a control 715, which is shown embedded within housing 710, in one embodiment only, as well as one or more sensors and one or more actuators. This control system of x-ray source assembly 700 includes functionality to actively control temperature of the anode stack relative to a setpoint temperature or voltage. The control system includes a heater to heat the anode stack in a heating mode when an anode stack temperature is below the setpoint temperature, and the control system switches to cooling the anode stack in a cooling mode when the anode stack temperature rises above the setpoint temperature. The setpoint temperature is selected to facilitate maintaining alignment between the anode stack and the optic. This enables the x-ray source assembly 700 to maintain a spot size 745 with stable intensity within a range of anode operating levels and modes.

FIG. 8 depicts one embodiment of a functional control loop, in accordance with an aspect of the present invention. As shown in FIG. 8, one or more sensors 801 provide feedback on, for example, temperature from tube/housing 830 (“T”), such as the anode stack temperature. The feedback is fed to a processor 810 (or other control components such as discussed below in connection with FIGS. 11 & 12) implementing the control function. By way of example, a control function can be implemented where a temperature offset is determined between the value from a temperature sensor (TS) and a setpoint or reference temperature (R) in order that the current position (K), rate of change (d/dt) and accumulated history (E) can be determined. The results of such a proportional integral derivative function can be obtained and summed to provide an output as a function of time (0(t)). This output is provided to one or more actuators 820 which effect an automatic change in anode temperature (“T”), thus maintaining the anode source spot location relative to the output structure. This monitoring and adjustment process can be continuously repeated by the control system of the x-ray source assembly.

In one embodiment, control operation can include using the continuous output of a PID type controller and actuating one or more individual temperature control elements, such as one or more heating elements (heaters) or one or more cooling elements (fans), to produce an accurate and timely response for overall thermal control. The overall thermal response is therefore uniform and finely controllable, avoiding discontinuous control actions and oscillatory limit cycles in overall system response.

Returning to FIG. 7, sensor/mode actuator 720 can include one or more temperature actuators physically coupled to base plate 165′. This temperature actuator(s) 720 can be, for example, any device for applying heat (in a heating mode) or applying cooling (in a cooling mode) to base plate 165′ to add/remove heat to/from the base plate. By way of example, the device can be a heating element such as a 10 Ohm power resistor, while an appropriate cooling element can be a forced air heat sink (e.g., fan) or a liquid based heat sink or other types of cooling methods. The temperature actuator(s) can be utilized during operation of the x-ray source assembly to maintain the anode x-ray spot at an optimum orientation with respect to one or more output structures such as the x-ray collection optic. The application of heat or removal of heat from the base plate is accomplished so that a consistent temperature at, or within a predefined range of, the setpoint is maintained across the anode stack throughout operation of the x-ray source assembly notwithstanding change in one or more operating conditions of the assembly.

Specifically, in one embodiment, thermal expansion of the base assembly and HV anode are maintained within a tolerance that enables the generated x-rays to be consistently aligned with, for example, the collection optic throughout the operating ranges of the x-ray source assembly. The addition of applied heat can occur, for example, when the x-ray source assembly shifts to a reduced operating power so that the HV anode and the base assembly elements do not undergo a reduction in size due to a reduced dissipation of heat therethrough, enabling an optimum alignment of x-rays and the collection optic to be maintained. In one embodiment, the heating element can be included internal to the base plate, while the cooling element can be thermally coupled to the exposed surface of the base plate. For instance, in one embodiment, the cooling element can be, or include, an air-cooled heat sink, such as a plurality of thermally conductive fins extending from the exposed surface of the base plate across which air flows, propelled by one or more fans of the control system.

FIG. 9 is a flowchart of one embodiment of processing which can be implemented by control 715 of FIG. 7. FIG. 9 represents a loop which is periodically repeated by the control during operation of the x-ray source assembly. This can for example, apply or remove heat from the base assembly in response to a change in one or more operating conditions, such as the power level applied to the anode, and thereby maintain a consistent average temperature across the anode stack and thus enable the emitted x-rays to be optimally aligned with respect to the input of the collection optic.

As shown in the embodiment of FIG. 9, control processing begins by reading the anode power level 900. In one embodiment, the anode power level can be determined from two analog inputs whose signals range, for example, between 0 and 10 V. One input communicates the voltage at which the power supply supplying power to e-gun 115 (FIG. 7) is operating, while a second input communicates the current being drawn by the power supply. From these two inputs, the power at which e-gun 115 is operating can be determined, which is also the power level of the anode.

Processing next reads the temperature of the anode stack as well as the source housing 910. As noted above, the temperature of the anode stack can be obtained from the base plate of the base assembly using a temperature sensor, with the resultant signal fed back to the processor embedded within the assembly. The housing temperature also can include a temperature sensor, which in one embodiment, is thermally coupled to a surface of the housing in order to measure expansion or contraction of the enclosure. The desirability of measuring housing temperature assumes that the optic or other output structure being monitored is mechanically coupled to the housing.

Next, control processing determines a reference or setpoint temperature for the read power level 920. The reference temperature is a desirable predetermined temperature for the anode stack at the measured anode power level. Reference temperatures can be determined during a calibration procedure for the x-ray source assembly, and can either be unique to a particular assembly or generic to a plurality of identically manufactured x-ray source assemblies. FIG. 10 depicts one embodiment of a table which can be employed in order to look up the reference temperature for a read power level. As shown, the table of FIG. 10 also employs the housing temperature as another operating condition to be considered in determining the desired reference or setpoint temperature for the anode stack. Thus, depending upon the housing temperature for the x-ray source assembly and the anode power level, a desired reference temperature for the anode stack is obtained.

In one embodiment, the reference temperature and the read temperatures can be fed to a position, rate and accumulated history control algorithm such as described above in connection with FIG. 8. The algorithm can be employed to calculate the outputs to the one or more actuators 930. One of ordinary skill in the art can readily implement a proportion integral derivative algorithm to accomplish this function. Once the output is obtained, the output is provided to the actuator(s) in order to, for example, maintain the anode source spot location relative to the optic input 940.

As one specific example, the control can output a signal which includes a pulse width modulated signal that enables the cooling fan to operate at a range of rotational speeds, and thereby remove heat at an appropriate rate from the base plate of the anode stack. The duty cycle of such a pulse width modulated output can be determined by the operating power of the anode. A second output could enable variation in the power supplied to the heating element, and thereby variations in the amount of heat added to the base plate of the anode stack. In one embodiment, the control, after performing the proportional integral differential (PID) algorithm, can utilize a formula or a look-up table to determine the temperature that the base plate of the anode stack should be maintained at (i.e., reference or setpoint temperature) for a particular power level at which the anode is currently operating.

In one or more implementations, control processing such as disclosed herein can control temperature of the anode stack by simultaneously both heating and cooling the anode stack in order to provide a greater degree of temperature control and to, for instance, avoid any overshoot of cooling or heating of the anode stack, such as upon starting the cooling device(s).

In one or more other embodiments, a control, or more generally a control system for an x-ray source assembly such as disclosed herein, operates, in part, to maintain intensity of the output x-rays during operation of the x-ray source assembly by actively controlling temperature of the anode stack relative to a setpoint or defined setpoint range in only one of a heating mode or a cooling mode at a time. In the heating mode, the control system heats the anode stack when the anode stack temperature is below the setpoint or defined setpoint range, and switches to the cooling mode to cool the anode stack when the anode stack temperature rises above the setpoint or defined setpoint range. In one implementation, a single setpoint (or defined setpoint range) is used, such as a single setpoint temperature or voltage, to which the heating or cooling are both controlled. In one embodiment, the setpoint (or defined setpoint range) can be calibrated, or predetermined, to facilitate maintaining alignment between the source spot of the anode stack and the optic, for instance, for a particular assembly operating power, as well as different ambient temperatures, etc. Advantageously, as disclosed herein, the control system is further configured to minimize cooling overshoot of the anode stack relative to the setpoint upon entering the cooling mode, as well as limiting heating overshoot of the anode stack relative to the setpoint in the heating mode. In an embodiment disclosed herein, the control system maintains the anode stack temperature within a setpoint range of ±0.1° C. of the particular setpoint temperature to which the assembly is being controlled.

In one or more embodiments, the control system includes a cooling device, such as a fan or a coolant pump, and the cooling system initiates operation of the cooling device to facilitate cooling the anode stack when the anode stack temperature rises above the setpoint. In one aspect, this initiating operation of the cooling device is configured to minimize cooling overshoot of the anode stack relative to the setpoint. For instance, in one embodiment, the cooling device requires a minimum power level to start, and once started, the control system lowers the power level to the cooling device to continue running the cooling device at a lower power level that the minimum power level required to start, thereby extending the operating range of the cooling device, and facilitating minimizing cooling overshoot with starting of the cooling device.

In one or more embodiments disclosed herein, the control system includes a kickstart pulse generator to facilitate generating a kickstart power signal for the cooling device (e.g., fan) sufficient to start the cooling device. In one embodiment, the control system also includes a pulse-width modulation (PWM) generator to generate a PWM power signal for the cooling device. Based on the cooling device being started by the kickstart pulse generator, the control system then provides the PWM power signal to drive the cooling device. In this manner, the cooling device can be driven at a lower power signal than required to start the power device, which minimizes potential cooling overshoot of the setpoint upon starting of the cooling device, to maintain the anode stack temperature within, for instance, ±0.1° C. of the reference or setpoint temperature.

In one or more implementations, upon entering the cooling mode, the control system inhibits the kickstart pulse generator from providing the kickstart power signal to the cooling device until the PWM power signal generated by the PWM generator is above a minimum duty cycle to maintain the cooling device operational once started. Further, in one embodiment, the control system can also inhibit the kickstart generator from providing the kickstart power signal to the cooling device once the cooling device is started.

In one or more embodiments, the control system facilitates transition of the anode stack between a standby mode of the x-ray source assembly and an operational mode of the x-ray source assembly, where in the standby mode, heat is applied to the anode stack to maintain the anode stack temperature within a predefined range of the setpoint, and in the operational mode, cooling is applied to the anode stack to maintain the anode stack temperature within the same, or a different predefined range of the setpoint (e.g., within ±0.1° C. of the setpoint). As noted, in one embodiment, the control system actively controls temperature of the anode stack by being in only one of the heating mode or the cooling mode of the control system at a time. This advantageously saves power, as well as extending longevity of the x-ray source assembly.

In one or more embodiments, the optic includes at least one of a focusing optic or a collimating optic. In one or more specific embodiments, the optic includes one of a polycapillary optic or a doubly-curved crystal.

Advantageously disclosed herein is a control system which eliminates the need for running both, for instance, a heater and a fan, during operation of an x-ray source assembly in order to provide sufficient temperature control over the anode stack in order to, for instance, facilitate maintaining intensity of the output x-rays during operation of the x-ray source assembly. Further, disclosed herein is a control system which eliminates any need for maintaining the anode stack powered between measurements such as in a standby mode, to maintain stable optic alignment and performance. This is accomplished by the control system maintaining, in a heating mode, temperature of the anode stack, without power being applied to the anode. This allows the x-ray tube to be ramped up once started, and between every measurement, the anode power is allowed to go to zero in standby mode. By not requiring the anode power to be ON in standby mode, the operational life of the x-ray source assembly is extended significantly.

Further, as described herein, the control system is configured to minimize cooling overshoot of the anode stack relative to the setpoint upon entering the cooling mode. For instance, in one embodiment, a power signal sufficient to start the cooling device (e.g., fan) is provided by a kickstart generator, and once the fan is started, the power signal is adjusted to be supplied via, a pulse-width modulation generator which has a duty cycle sufficient to maintain operation of the cooling device, but a lower power signal perhaps than actually required to start the cooling device. In one embodiment, the control system provides a power signal of sufficient duration to let the current reach a level within the cooling device to start the cooling device, and once started, the control system reverts to a different power signal with, for instance, a high frequency, such as, for instance, a 25 kHz signal.

In one or more implementations, the control system processing disclosed herein can be implemented digitally as program code, which can include software and/or hardware. For instance, program code in certain embodiments of the present invention can include fixed function hardware, while other embodiments can utilize a software-based implementation of the functionality described. Certain embodiments can combine both types of program code. Embodiments of the present invention can include a computer-implemented method, a system, and/or a computer program product, where program code executing on one or more processors performs the control processing disclosed herein.

In one or more other implementations, the control processing of a control system such as disclosed herein can be implemented as an analog control circuit. FIGS. 11 & 12 depict embodiments of such an analog-implemented control system.

As noted, FIG. 11 depicts one embodiment of a control system 1100, and control system processing, for an x-ray source assembly, in accordance with one or more aspects of the present invention. As illustrated, one or more temperature sensors 1102 are associated with the x-ray source assembly, such as attached or coupled to the anode stack, to sense temperature of the anode stack and provide a voltage output representative of the sensed temperature. In one embodiment, this voltage output can be amplified 1104 and provided to both an under-temperature difference amplifier circuit 1110, and an cooling device difference amplifier circuit 1120 for comparison against a pre-calibrated setpoint voltage (or temperature) 1106.

In one or more embodiments, under-temperature difference amplifier 1110 powers a heater driver 1112, such as a variable output voltage regulator, which drives one or more heaters 1114. Note that heater(s) 1114 can be, in one or more implementations, a resistive heater coupled to or otherwise associated with the anode stack, and located to provide heating to the anode stack in order to maintain temperature of the anode stack at, or near, the setpoint calibrated for the particular power level of anode stack operation. For instance, in one embodiment, the heater(s) is coupled to base plate 165′ in the x-ray source assembly embodiment of FIG. 7, described above.

In the embodiment shown, over-temperature difference amplifier 1120 provides a signal to a pulse-width modulation (PWM) duty cycle generator 1122, which along with a PWM frequency generator 1124, provides a PWM power signal to power one or more anode stack cooling devices 1130, such as one or more fans providing airflow across the anode stack heat sink, such as across a plurality of thermally conductive fins (not shown) extending from base plate 165′ in the x-ray source assembly embodiment of FIG. 7. Note that in one or more other embodiments, the anode stack cooling device could be one or more coolant pumps controlling liquid coolant flow through one or more liquid-cooled cold plates coupled to the anode stack base plate.

In the embodiment illustrated, a kickstart pulser or pulse generator 1126 is also provided to provide a kickstart power signal to power anode stack cooling device 1130 when difference amplifier 1120 initially detects an over-temperature condition and the anode stack cooling device is to be started. As discussed herein, in one embodiment, the kickstart pulse generator provides a kickstart power signal sufficient to start the cooling device. Where the cooling device includes a rotor, the kickstart power signal is sufficient to overcome the stiction of the bearings and the inertia of the rotor and blades. Once started, the control system provides the PWM power signal to then control power to the cooling device. Further, in one embodiment, a minimum duty cycle (MDC) inhibit circuit 1128 is also provided to prevent powering of the anode stack cooling device below a duty cycle required to maintain the cooling device in an ON state. As an optional embodiment, a rotational-sensed, pulse-inhibit circuit 1140 can be provided to block the kickstart pulser from providing the kickstart power signal once the PWM power signal is established and provided to the anode stack cooling device. As explained herein, in one embodiment, the PWM power signal can advantageously be a lower power signal where desirable than the kickstart power signal required to start the cooling device, thereby allowing the cooling device to operate in a lower power region, where less cooling is provided to the anode stack. This advantageously extends the low cooling range of the cooling device to, for instance, minimize cooling overshoot of the setpoint upon entering the cooling mode of the control system where less cooling is needed to maintain the anode stack temperature within the desired range (e.g., ±0.1° C. of setpoint).

FIG. 12 depicts further details of one embodiment of a control system, such as control system 1100 of FIG. 11. As noted, and by way of example only, FIG. 12 depicts one embodiment of an analog implementation of a control system for an x-ray source assembly, in accordance with one or more aspects of the present invention. As illustrated, an anode temperature sensor and amplifier 1200 is provided which includes, for instance, one or more temperature sensors coupled to the anode stack, such as integrated within or attached to the thermally conductive base of the anode stack assembly. In one embodiment, the temperature sensor provides a 1 mV/° C. sensor output, and the amplifier provides a 10× amplification to produce a scaling of 100 mV/° C. A single setpoint, such as a setpoint temperature or voltage 1106, is predetermined for the control system, for instance, through prior calibration of the control system and the x-ray source assembly operation for a given power level, ambient temperature, etc. This single setpoint voltage 1106 in FIG. 12 controls the operation of both the heating mode and the cooling mode of the control system. In one example, a setpoint of 4 V can correspond to an anode stack temperature of 40° C. Note that in one or more other embodiments, different setpoints can be used for controlling the heating mode and cooling mode, if desired.

As illustrated in FIG. 12, the under-temperature difference amplifier (or heater-difference amplifier) 1110 receives as input the amplified temperature value and the setpoint voltage value, for instance, across resistors R2, R3, which in one embodiment can be 10 k resistors, with resistor R1 being, for instance, a 100 k Ohm resistor. Heater power driver 1112 is, in one embodiment, a variable output voltage regulator which drives heater 1114. Further, in one embodiment, resister R4 is 100 k Ohm, R5 is 1 k Ohm, and R6 is 20 k Ohm with, for instance, the heater being a 150 k Ohm heater, by way of example only. The span from zero power to full heater drive is determined by the gain of the heater-difference amplifier 1110 and the Vref of the heater driver buck regulator. In this example, a V_{ref} of 1 volt results in a span of 100 mV (1° C.) from full heater power to zero power. Within this 100 mV span, the heater power increases with the square of the deviation below setpoint, up to the maximum output.

When the x-ray tube is powered and generating heat, the anode stack temperature goes above the setpoint, and the control system switches from the heating mode to the cooling mode, i.e., the heater is switched OFF, and the cooling device is turned ON. In the cooling mode, the cooling device is driven with a variable duty cycle pulse-width modulation (PWM) power signal from PWM generator 1122. As noted, in one embodiment, the circuitry of FIG. 12 can be implemented with analog components, requiring no software program code, if desired. In one implementation, a 555 timer generates a sawtooth waveform between a lower threshold of 4V and an upper threshold of 8V. The cooling device difference amplifier 1120 is referenced in the circuit to the upper sawtooth threshold (UST) of 8V. When the sensed anode stack temperature goes above the setpoint, the decreasing output of the inverting difference amplifier crosses 8V, or zero percent duty cycle. When the output goes to 4V, the duty cycle is 100%. The temperature span between zero percent and 100% duty cycle is 4V (8V−4V=4V)/the gain of the cooling device difference amplifier (1 mg/10 k=100), or 40 mV, in one embodiment. More particularly, in one embodiment, resistors R7 & R10 can have a 1 k Ohm resistance, and resistors R8 & R9 can have a 10 k Ohm resistance. Further, resistor R11 can be, in one embodiment, 100 k Ohms.

In one embodiment, the usable span is less than 40 mV, since the cooling devices (e.g., fans) will not start at a very low duty cycle. Cooling devices trying to start, but not starting, can make disturbing noises, and so it is desirable to inhibit the PWM power signal below some minimum duty cycle at the startup. Further, a cooling device, such as a fan, starting operation at the minimum duty cycle can remove heat quickly, and may tend to overshoot the setpoint, cycling OFF and ON, as a result.

As disclosed herein, extending the operating range of the cooling device (for instance, a fan) is desirable for minimizing the temperature fluctuations around the setpoint, as well as reducing audible fan speed variations. Advantageously, the duty cycle to sustain running of a cooling device is considerably lower than the minimum duty cycle required for starting the fan. In other words, once started, the control system can switch the duty cycle of the power signal, driving the cooling device with a lower duty cycle once the cooling device is started.

More particularly, in one embodiment, the cooling device is driven when the PWM input is high. Resistor R12 (e.g., 1 k Ohm) pulses input high, but either the minimum duty cycle (MDC) comparator 1128, or a pulse-width modulation (PWM) comparator 1210, can pull the power signal down. In one embodiment, the MDC and PWM comparators 1128, 1210, are open collector-type, and can only pull down, that is, they cannot pull the signal up. The output of the cooling device difference amplifier 1120 is connected to the PWM comparator 1210 via resistor R11. In one embodiment, the difference between the 100 mV/° C. scaled sensed temperature and the setpoint temperature is amplified by a factor of 100.

As noted, a kickstart pulse generator is provided to provide a kickstart power signal sufficient to start the cooling device. For instance, when the kickstart pulse goes low, it drives the inverting (−) input of the PWM comparator 1210 below the non-inverting (+) input. This turns OFF the output transistor of the comparator, and allows the output to be pulled up, but this will only happen when the MDC comparator 1128 output is also OFF. Because diode D1 is provided in the connection, the kickstart pulse generator 1126 can only pull the signal down. Further, because of resistor R11, the kickstart pulse generator 1126 does not change the output of the cooling device difference amplifier 1120 going to the MDC comparator 1128.

The kickstart pulse generator can operate continuously, but is disabled by the MDC comparator 1128 once the cooling device is started, in one embodiment. Below the MDC, the MDC comparator 1128 disables the cooling device drive by holding the cooling device PWM input low. Below MDC, neither the PWM comparator, nor the kickstart generator, can activate the anode stack cooling device 1130.

The minimum duty cycle is controlled by a voltage which, in this case, is between zero percent duty cycle of 8V, and the 100% duty cycle of 4V. This is a linear relationship where: MDC voltage of 6V=50% duty cycle, MDC voltage of 7V=25% duty cycle, and MDC voltage of 7.5V=12.5% duty cycle. The optimal minimum duty cycle (MDC) is specific to the cooling device used, although cooling devices of similar size and specifications could operate with the same minimum duty cycle.

As noted, in one or more implementations, the kickstart pulse generator 1126 outputs a low frequency of pulses of sufficient duration to overcome the stiction and inertia to start the cooling device (e.g., fan) turning. This allows a lower duty cycle signal to then be used to power the turning cooling device. The low frequency pulse provided by the kickstart pulse generator can either be applied continually to the anode stack cooling device (PWM input), or can be turned OFF with feedback from a tachometer signal provide by the cooling device. As noted, the kickstart pulse generator works by pulling down the PWM comparator inverting input below the PWM sawtooth on the non-inverting input, via diode D1.

Those skilled in the art will note that provided herein is an enhanced control system for an x-ray source assembly. In operation, from a cold start to measurement mode, the control system is in a heating mode, with, for instance, the heater operated full ON, and the beam ramping up. As anode stack temperature rises towards the setpoint, the heater is controllably turned down, then OFF, and the control system switches to the cooling mode as temperature rises above the setpoint, where one or more cooling devices, such as one or more fans, are turned ON to facilitate regulating the anode stack temperature while the beam is operational. In transitioning the x-ray source assembly from operating to standby mode, the beam is ramped to zero power, and the cooling device slows down, then stops once temperature drops below the setpoint. At this time, the control system switches to heating mode, with the heater supplying an amount of power sufficient to maintain temperature of the anode stack. From a standby mode of the x-ray source assembly to a full operational and measurement mode, the power to the anode is ramped up to a measurement power level, and the control system enters the cooling mode upon sensed temperature exceeding the setpoint. Upon entering the cooling mode, the cooling device speed is slowly increased to maintain the setpoint temperature. Note that this can include using the kickstart signal to turn the cooling device ON, then using the PWM signal to drive the cooling device at a lower duty cycle signal (i.e., lower power signal) for a time than required to start the cooling device.

By way of further example, FIG. 13 depicts a computer system 1300 in communication with external device(s) 1312, which can be used to implement one or more control aspects disclosed herein. Computer system 1300 includes one or more processor(s) 1302, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1302 can also include one or more registers to be used by one or more of the functional components. Computer system 1300 also includes a memory 1304, input/output (I/O) devices 1308, and I/O interfaces 1310, which may be coupled to processor(s) 1302 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and Peripheral Component Interconnect (PCI).

Memory 1304 can be, or include, main or system memory (e.g. Random Access Memory) used in the execution of program instructions, a storage device(s) such as hard drive(s), flash media, or optical media as examples, and/or cache memory, as examples. Memory 1304 can include, for instance, a cache, such as a shared cache, which may be coupled to local caches (examples include L1 cache, L2 cache, etc.) of processor(s) 1302. Additionally, memory 1304 can be, or include, at least one computer program product having a set (e.g., at least one) of program modules, instructions, code or the like that is/are configured to carry out functions of embodiments described herein when executed by one or more processors.

Memory 1304 can store an operating system 1305 and other computer programs 1306, such as one or more computer programs/applications that execute to perform aspects described herein. Specifically, programs/applications can include computer readable program instructions that can be configured to carry out functions of embodiments of aspects described herein.

Examples of I/O devices 1308 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device can be incorporated into the computer system as shown, though in some embodiments an I/O device can be regarded as an external device (1312) coupled to the computer system through one or more I/O interfaces 1310.

Computer system 1300 can communicate with one or more external devices 1312 via one or more I/O interfaces 1310. Example external devices include a keyboard, a display, one or more data sensors, and/or any other devices that (for instance) enable a user to interact with computer system 1300. Other example external devices include any device that enables computer system 1300 to communicate with one or more other computing systems or peripheral devices. A network interface/adapter is an example I/O interface that enables computer system 1300 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems. (BLUETOOTH® is a registered trademark of Bluetooth SIG, Inc., Kirkland, Wash., U.S.A.)

Communication between I/O interfaces 1310 and external devices 1312 can occur across wired and/or wireless communications link(s) 1311, such as Ethernet-based wired or wireless connections. Example wireless connections include cellular, Wi-Fi, Bluetooth®, proximity-based, near-field, or other types of wireless connections. More generally, communications link(s) 1311 can be any appropriate wireless and/or wired communication link(s) for communicating data between systems and/or devices to facilitate one or more aspects disclosed herein.

A particular external device(s) 1312 can include one or more data storage devices, which can store one or more programs, one or more computer readable program instructions, and/or data, etc. Computer system 1300 can include and/or be coupled to and in communication with (e.g. as an external device of the computer system) removable/non-removable, volatile/non-volatile computer system storage media. For example, it can include and/or be coupled to a non-removable, non-volatile magnetic media (typically called a “hard drive”), a magnetic disk drive for reading from and writing to a removable, non-volatile magnetic disk, and/or an optical disk drive for reading from or writing to a removable, non-volatile optical disk, such as a CD-ROM, DVD-ROM or other optical media.

Computer system 1300 can be operational with numerous other general purpose or special purpose computing system environments or configurations. Computer system 1300 can take any of various forms, well-known examples of which include, but are not limited to, personal computer (PC) system(s), server computer system(s), thin client(s), thick client(s), workstation(s), laptop(s), handheld device(s), mobile device(s)/computer(s), such as smartphone(s), tablet(s), and wearable device(s), multiprocessor system(s), microprocessor-based system(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

As will be appreciated by one skilled in the art, control aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system”. Furthermore, control aspects of the present invention may take the form of a computer program product embodied in one or more computer readable medium(s) having computer readable program code embodied thereon.

Any combination of one or more computer readable medium(s) can be utilized. The computer readable medium can be a computer readable signal medium or a computer readable storage medium. A computer readable signal medium can be any non-transitory computer readable medium that is not a computer readable storage medium and that can communicate, propagate, or transport a program for use by or in connection with an instruction execution system, apparatus or device.

A computer readable storage medium can be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium is any tangible medium that can contain or store a program for use by or in connection with an instruction execution system, apparatus, or device.

In one example, a computer program product includes, for instance, one or more computer readable storage media to store computer readable program code means or logic thereon to provide and facilitate one or more aspects of the present invention.

Program code embodied on a computer readable medium can be transmitted using an appropriate medium, including but not limited to wireless, wireline, optical fiber cable, RF, etc., or any suitable combination of the foregoing.

Computer program code for carrying out control and/or calibration operations for aspects of the present invention may be written in any combination of one or more programming languages, including an object oriented programming language, such as Java, Smalltalk, C++ or the like, and conventional procedural programming languages, such as the “C” programming language, assembler or similar programming languages. The program code may execute entirely on the user's computer, partly on the user's computer, on the user's personal device (e.g., phone, tablet, wearable), as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to block diagrams of methods, apparatus (systems) and computer program products according to embodiments of the invention. It will be understood that the control block of the diagram can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions can also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions can also be loaded onto a computer, other programmable data processing apparatus (e.g., mobile device/phone), or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The block diagram in the figure illustrates the architecture, functionality, and operation of possible implementations of systems, methods and computer program products according to various embodiments of the present invention. In this regard, one or more blocks in the diagram can represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession can, in fact, be executed substantially concurrently, or the blocks may sometimes be executed in the reverse order, depending upon the functionality involved. It will also be noted that one or more blocks of the diagram can be implemented by special purpose hardware-based systems that perform the specified functions or acts, or combinations of special purpose hardware and computer instructions.

In one aspect of the present invention, an application can be deployed for performing one or more aspects of the present invention. As one example, the deploying of an application comprises providing computer infrastructure (including, e.g., internet/cloud/IOT resources and/or a mobile device) operable to perform one or more aspects of the present invention.

As a further aspect of the present invention, a computing infrastructure can be deployed comprising integrating computer readable code into a computing system, in which the code in combination with the computing system is capable of performing one or more aspects of the present invention.

As yet a further aspect of the present invention, a process for integrating computing infrastructure comprising integrating computer readable code into a computer system can be provided. The computer system includes a computer readable medium, in which the computer medium comprises one or more aspects of the present invention. The code in combination with the computer system is capable of performing one or more aspects of the present invention.

Although various embodiments are described above, these are only examples. For example, computing environments of other architectures can incorporate and use one or more aspects of the present invention. Additionally, the network of nodes can include additional nodes, and the nodes can be the same or different from those described herein. Also, many types of communications interfaces may be used.

Further, a data processing system suitable for storing and/or executing program code is usable that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Input/Output or I/O devices (including, but not limited to, mobile device/phone, keyboards, displays, pointing devices, DASD, tape, CDs, DVDs, thumb drives and other memory media, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards are just a few of the available types of network adapters.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. An x-ray source assembly comprising:
an anode stack having a source spot upon which electronics impinge based on power supplied to the assembly; and
a control system to facilitate maintaining intensity of output x-rays during operation of the x-ray source assembly, the control system to actively control temperature of the anode stack relative to a setpoint or defined setpoint range, wherein the control system heats the anode stack in a heating mode when an anode stack temperature is below the setpoint or defined setpoint range, and switches to a cooling mode to cool the anode stack when the anode stack temperature rises above the setpoint or defined setpoint range.

2. The x-ray source assembly of claim 1, wherein the control system comprises a cooling device, the control system initiating operation of the cooling device to facilitate cooling the anode stack when the anode stack temperature rises above the setpoint or defined setpoint range, and wherein the initiating operation of the cooling device is configured to minimize cooling overshoot of the anode stack relative to the setpoint or defined setpoint range.

3. The x-ray source assembly of claim 2, wherein the cooling device requires a minimum power level to start, and once started, the control system lowers a power level to the cooling device to continue running the cooling device at a lower power level than the minimum power level required to start the cooling device, to minimize cooling overshoot of the anode stack with starting of the cooling device.

4. The x-ray source assembly of claim 2, wherein the control system further comprises a kickstart pulse generator to facilitate generating a kickstart power signal to power the cooling device sufficient to start the cooling device.

5. The x-ray source assembly of claim 4, further comprising a pulse-width modulation (PWM) generator to generate a PWM power signal to power the cooling device, wherein based on the cooling device being started by the kickstart pulse generator, the control system provides the PWM power signal to drive the cooling device.

6. The x-ray source assembly of claim 5, wherein upon entering cooling mode, the control system inhibits the kickstart pulse generator from providing the kickstart power signal to power the cooling device until the PWM power signal generated by the PWM generator is above a minimum duty cycle to maintain the cooling device operational once started.

7. The x-ray source assembly of claim 5, wherein the control system further inhibits the kickstart generator from providing the kickstart power signal to the cooling device once the cooling device is started.

8. The x-ray source assembly of claim 1, wherein the control system facilitates transition of the anode stack between a standby mode of the x-ray source assembly and an operational mode of the x-ray source assembly, wherein in the standby mode heat is applied to the anode stack to maintain the anode stack temperature within a predefined range of the setpoint, and in the operational mode cooling is applied to the anode stack to maintain the anode stack temperature within another predefined range of the setpoint.

9. The x-ray source assembly of claim 1, wherein the control system actively controls temperature of the anode stack by being in only one of the heating mode or the cooling mode of the control system at a time.

10. The x-ray source assembly of claim 1, further comprising an optic coupled to receive divergent x-rays generated at the source spot and transmit the output x-rays from the assembly, wherein the optic comprises at least one of a focusing optic, a collimating optic, or other redirecting optic.

11. The x-ray source assembly of claim 10, further comprising an optic coupled to receive divergent x-rays generated at the source spot and transmit the output x-rays from the assembly, wherein the optic comprises one of a polycapillary optic, a doubly-curved crystal, or other x-ray manipulating optic.

12. A method of fabricating an x-ray source assembly, the method comprising:
providing an anode stack having a source spot upon which electrons are to impinge based on power supplied to the x-ray source assembly; and
providing a control system to facilitate maintaining intensity of output x-rays during operation of the x-ray source assembly, the control system to actively control temperature of the anode stack relative to a setpoint or defined setpoint range, wherein the control system heats the anode stack in a heating mode when an anode stack temperature is below the setpoint or defined setpoint range, and switches to a cooling mode to cool the anode stack when the anode stack temperature rises above the setpoint or defined setpoint range.

13. The method of claim 12, wherein the control system comprises a cooling device, the control system initiating operation of the cooling device to facilitate cooling the anode stack when the anode stack temperature rises above the setpoint or defined setpoint range, wherein the initiating operation of the cooling device is configured to minimize cooling overshoot of the anode stack relative to the setpoint or defined setpoint range.

14. The method of claim 13, wherein the cooling device requires a minimum power level to start, and once started, the control system lowers a power level to the cooling device to continue running the cooling device at a lower power level than the minimum power level required to start the cooling device, to minimize cooling overshoot of the anode stack with starting of the cooling device.

15. The method of claim 13, wherein the control system further comprises a kickstart pulse generator to facilitate generating a kickstart power signal to power the cooling device sufficient to start the cooling device.

16. The method of claim 15, further comprising a pulse-width modulation (PWM) generator to generate a PWM power signal to power the cooling device, wherein based on the cooling device being started by the kickstart pulse generator, the control system provides the PWM power signal to drive the cooling device.

17. The method of claim 16, wherein upon entering the cooling mode, the control system further inhibits the kickstart pulse generator from providing the kickstart power signal to the cooling device until the PWM power signal generated by the PWM generator is above a minimum duty cycle to maintain the cooling device operational once started.

18. The method of claim 16, wherein the control system further inhibits the kickstart generator from providing the kickstart power signal to the cooling device once the cooling device is started.

19. The method of claim 12, wherein the control system facilitates transition of the anode stack between a standby mode of the x-ray source assembly and an operational mode of the x-ray source assembly, wherein in the standby mode heat is applied to the anode stack to maintain the anode stack temperature within a predefined range of the setpoint, and in the operational mode cooling is applied to the anode stack to maintain the anode stack temperature within another predefined range of the setpoint.

20. The method of claim 12, wherein the control system actively controls temperature of the anode stack by being in only one of the heating mode or the cooling mode of the control system at a time.
